SEQUENCE LISTING (15 sequences)

SEQ ID NO: 1 - protein, 118 aa, Artificial Sequence
  An artificial amino acid sequence of the heavy chain variable region of the anti CD87 monoclonal antibody hCD87scFv of the chimeric antigen receptor hCD87-CAR
GVKLQQSGPEVVKPGASVKISCKASGYSFTNFYIHWVKQRPGQGLEWIGWIFHGSDNTEYNEKFKDKATLTADTSSSTAYMQLSSLTSEDSAVYFCARWGPHWYFDVWGQGTTVTVSS

SEQ ID NO: 2 - protein, 106 aa, Artificial Sequence
  An artificial amino acid sequence of the light chain variable region of the anti CD87 monoclonal antibody hCD87scFv of the chimeric antigen receptor hCD87-CAR
DIVLTQSPDITAASLGQKVTITCSASSSVSYMHWYQQKSGTSPKPWIFEISKLASGVPARFSGSGSGTSYSLTISSMEAEDAAIYYCQQWNYPFTFGGGTKLEIKR

SEQ ID NO: 3 - DNA, 354 bp, Artificial Sequence
  An artificial nucleic acid sequence encoding the heavy chain variable region of the anti CD87 monoclonal antibody hCD87scFv of the chimeric antigen receptor hCD87-CAR
ggcgtgaagctgcagcagagcggccccgaggtggtgaagcccggcgccagcgtgaagatcagctgcaaggccagcggctacagcttcaccaacttctacatccactgggtgaagcagcgccccggccagggcctggagtggatcggctggatcttccacggcagcgacaacaccgagtacaacgagaagttcaaggacaaggccaccctgaccgccgacaccagcagcagcaccgcctacatgcagctgagcagcctgaccagcgaggacagcgccgtgtacttctgcgcccgctggggcccccactggtacttcgacgtgtggggccagggcaccaccgtgaccgtgagcagc

SEQ ID NO: 4 - DNA, 318 bp, Artificial Sequence
  An artificial nucleic acid sequence encoding the light chain variable region of the anti CD87 monoclonal antibody hCD87scFv of the chimeric antigen receptor hCD87-CAR
gacatcgtgctgacccagagccccgacatcaccgccgccagcctgggccagaaggtgaccatcacctgcagcgccagcagcagcgtgagctacatgcactggtaccagcagaagagcggcaccagccccaagccctggatcttcgagatcagcaagctggccagcggcgtgcccgcccgcttcagcggcagcggcagcggcaccagctacagcctgaccatcagcagcatggaggccgaggacgccgccatctactactgccagcagtggaactaccccttcaccttcggcggcggcaccaagctggagatcaagcgc

SEQ ID NO: 5 - protein, 83 aa, Artificial Sequence
  An artificial amino acid sequence of the CD8a hinge region of the chimeric antigen receptor hCD87-CAR
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 6 - DNA, 249 bp, Artificial Sequence
  An artificial nucleic acid sequence of the CD8a hinge region of the chimeric antigen receptor hCD87-CAR
ttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaac

SEQ ID NO: 7 - protein, 21 aa, Artificial Sequence
  An artificial amino acid sequence of the CD8a signal peptide of the amino terminal of the anti CD87 monoclonal antibody hCD87scFv
MALPVTALLLPLALLLHAARP

SEQ ID NO: 8 - DNA, 63 bp, Artificial Sequence
  An artificial nucleic acid sequence of the CD8a signal peptide of the amino terminal of the anti CD87 monoclonal antibody hCD87scFv
atggccttaccagtgaccgccttgctcctgccgctggccttgctgctccacgccgccaggccg

SEQ ID NO: 9 - protein, 15 aa, Artificial Sequence
  An artificial amino acid sequence of the connecting peptide between the heavy chain variable region and the light chain variable region of the anti CD87 monoclonal antibody hCD87scFv
GGGGSGGGGSGGGGS

SEQ ID NO: 10 - DNA, 45 bp, Artificial Sequence
  An artificial nucleic acid sequence of the connecting peptide between the heavy chain variable region and the light chain variable region of the anti CD87 monoclonal antibody hCD87scFv
ggtggtggtggttctggtggtggtggttctggcggcggcggctcc

SEQ ID NO: 11 - protein, 1096 aa, Artificial Sequence
  An artificial amino acid sequence of the chimeric antigen receptor hCD87-CAR
MALPVTALLLPLALLLHAARPGVKLQQSGPEVVKPGASVKISCKASGYSFTNFYIHWVKQRPGQGLEWIGWIFHGSDNTEYNEKFKDKATLTADTSSSTAYMQLSSLTSEDSAVYFCARWGPHWYFDVWGQGTTVTVSSGGGGSGGGGSGGGGSDIVLTQSPDITAASLGQKVTITCSASSSVSYMHWYQQKSGTSPKPWIFEISKLASGVPARFSGSGSGTSYSLTISSMEAEDAAIYYCQQWNYPFTFGGGTKLEIKRGSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSVPGVGVPGVGARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 12 - DNA, 3291 bp, Artificial Sequence
  An artificial nucleic acid sequence of the chimeric antigen receptor hCD87-CAR
atggccttaccagtgaccgccttgctcctgccgctggccttgctgctccacgccgccaggccgggcgtgaagctgcagcagagcggccccgaggtggtgaagcccggcgccagcgtgaagatcagctgcaaggccagcggctacagcttcaccaacttctacatccactgggtgaagcagcgccccggccagggcctggagtggatcggctggatcttccacggcagcgacaacaccgagtacaacgagaagttcaaggacaaggccaccctgaccgccgacaccagcagcagcaccgcctacatgcagctgagcagcctgaccagcgaggacagcgccgtgtacttctgcgcccgctggggcccccactggtacttcgacgtgtggggccagggcaccaccgtgaccgtgagcagcggtggtggtggttctggtggtggtggttctggcggcggcggctccgacatcgtgctgacccagagccccgacatcaccgccgccagcctgggccagaaggtgaccatcacctgcagcgccagcagcagcgtgagctacatgcactggtaccagcagaagagcggcaccagccccaagccctggatcttcgagatcagcaagctggccagcggcgtgcccgcccgcttcagcggcagcggcagcggcaccagctacagcctgaccatcagcagcatggaggccgaggacgccgccatctactactgccagcagtggaactaccccttcaccttcggcggcggcaccaagctggagatcaagcgcggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcagagccgagggcaggggaagtcttctaacatgcggggacgtggaggaaaatcccgggcccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 13 - DNA, 1671 bp, Artificial Sequence
  An artificial nucleic acid sequence of the IL12 region and the connection peptide of the chimeric antigen receptor hCD87-CAR
agagccgagggcaggggaagtcttctaacatgcggggacgtggaggaaaatcccgggcccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 14 - DNA, 25 bp, Artificial Sequence
  An artificial nucleic acid sequence of the upstream primer used for amplifying a fragment of the hCD8 7scFv cDNA obtained by reverse transcribing the RNA product of hCD87-CAR gene structure expression
ccgccttgctcctgccgctggcctt

SEQ ID NO: 15 - DNA, 25 bp, Artificial Sequence
  An artificial nucleic acid sequence of the downstream primer used for amplifying a fragment of the hC D87scFv cDNA obtained by reverse transcribing the RNA product of hCD87-CAR gene structure expression
caggaagaccggcacgaaggatccg

(19) United States
(12) Patent Application Publication
(10) Pub. No.: US 2017/0145106 A1
(43) Pub. Date: May 25, 2017

(54) Chimeric antigen receptor hCD87-CAR and applications thereof

(71) Applicants: Renzhi Wang, Beijing (CN); Yuan Ren, Beijing (CN)

(72) Inventors: Renzhi Wang, Beijing (CN); Yuan Ren, Beijing (CN)

(21) Appl. No.: 15/360,904

(22) Filed: Nov. 23, 2016

(30) Foreign Application Priority Data
Nov. 25, 2015 (CN) 201510829241.1

(51) Int. Cl. C07K 16/28; C12N 15/86; C12N 7/00

(52) U.S. Cl. CPC C07K 16/2896; C12N 7/00; C12N 15/86; C07K 2319/00; C07K 2317/24; C07K 2317/622; C07K 2317/53; C07K 2319/02; A61K 48/00

(57) ABSTRACT

The present invention relates to a cellular immunotherapy for treatment of tumors, particularly to a peptide of a chimeric antigen receptor hCD87-CAR and the applications thereof, the peptide of said antigen receptor being carried by a vector packaged in a lentivirus. The chimeric antigen receptor hCD87-CAR contains a fragment of anti CD87 monoclonal antibody hCD87scFv consisting of a sequence selected from a peptide comprising: (a) SEQ ID NO. 1 or SEQ ID NO. 2; (b) a peptide having the same function as the sequence of (a); and (c) a peptide having at least 90% homology to the sequence of (a). The hCD87-CAR is used for modifying T lymphocytes, and the modified T cells (CAR-T cells) can be used for the therapy of tumors that are cell surface CD87-positive.

### RELATED APPLICATION

The present application claims priority from the Chinese application number 201510829241.1, filed Nov. 25, 2015, the content of which is incorporated by reference in its entirety.

### FIELD OF THE INVENTION

The present invention relates to a cellular immunotherapy for treatment of tumors, particularly to a chimeric antigen receptor hCD87-CAR and applications thereof, the gene sequence of the chimeric antigen receptor being incorporated into a plasmid packaged into a lentivirus.

### BACKGROUND OF THE INVENTION

Pituitary adenoma is a common intracranial benign tumor, comprising about 10%-15% of brain tumors. There is a trend of increased occurrences of pituitary adenoma in recent years, which usually occurs in the young and middle-aged population. While non-function pituitary adenoma (NFPA) often grows large in size leading to blindness and other symptoms, abnormal secretion of hormones by functional pituitary adenomas can cause increased level of hormones such as prolactin (PRL), adrenocorticotropic hormone (ACTH), symptomized by swelling face, fat areas near the base of the neck, acne, hypertrichosis, hypertension, diabetes mellitus, osteoporosis, etc. In the female patients, it may result in miscarriage, infertility, menstrual disorders, non-pregnant galactorrhea, etc.

For a patient suffering from pituitary adenoma, the tumor cells in the patient, when diagnosed, have become predominant so that the patient's body's anti-tumor function has lost balance. In such case, the professional antigen-presenting cells (APC) such as dendritic cells in the immune system are often damaged to the extent that results in declining efficiency of the activating T cells for APC, further resulting in deficiency in their capacity and accuracy when attacking tumor cells. Moreover, tumor cells have the capability to escape from attacks by immune cells through a mechanism namely molecular escape due to the low- or non-expressed MHC genes. Thus, it is necessary to have an anti-tumor immunotherapy that not only overcomes insufficiency of the MHC-mediated cellular immunity, but also is capable of specifically targeting and killing tumor cells. The cellular immunotherapy CAR-T using a chimeric antigen receptor thus emerged.

By exogenous gene transduction technology, the CAR-T cellular immunotherapy enables a fusion protein of a single-chain antibody fragment (scFv) recognizing the tumor-specific antigen and a T cell activation sequence to be expressed on the surface of T cells such that scFv can be amplified in the patients' bodies by coupling with the signaling area of intracellular activation proliferation through its transmembrane portion, thereby exhibiting strong anti-tumor effects.

### SUMMARY OF THE INVENTION

The present invention provides a peptide of a chimeric antigen receptor namely hCD87-CAR, wherein the chimeric antigen receptor contains a fragment of an anti CD87 monoclonal antibody namely hCD87scFv that consists of a sequence selected from:

(a) SEQ ID NO. 1 or SEQ ID NO. 2;

(b) a sequence having the same function as the sequence of (a); and

(c) a sequence having at least 90% homology to the sequence of (a).

The present invention also provides a nucleic acid sequence, wherein the nucleic acid encodes a peptide of hCD87scFv.

The present invention further provides a plasmid and/or a lentivirus comprising the peptide of hCD87scFv.

The present inventor also further provides a method of preparing CD87-CAR-T cells and a method of preparing a pharmaceutical composition for tumor therapy by using the above peptide.

The present invention will be further described and illustrated as the following in conjunction with the drawings and examples provided herein.

### BRIEF DESCRIPTION OF DRAWINGS

FIG. 1 is a schematic view of the structure of the chimeric antigen receptor hCD87-CAR of the present invention;

FIG. 2 is a schematic view of the structure of the chimeric antigen receptor hCD87-CAR-IL12 of the present invention;

FIG. 3 is a confocal microscopy image showing the infection efficiency of Lenti-hCD87-CAR of the present invention to T cells;

FIG. 4A is a confocal microscope image showing the transfection rate of a plasmid to T cells; wherein the ordinate is the transfection rate, 2 D represents a two-dimensional image of confocal microscope;

FIG. 4B is a confocal microscope image showing the transfection rate of a plasmid to T cells; wherein the ordinate is the transfection rate, Z-Stack is a three-dimensional fluorescence image.

FIG. 5: 5A (also called FIG. 5A) is a microscopic observation chart showing the inducing migration actions of the primary prolactinoma (PRL) tumor cells, primary non-function pituitary adenomas (NFPA) tumor cells, and primary pituitary adrenalotropic hormone adenoma (Cushing) tumor cells among the pituitary adenomas respectively on hCD87-CAR-T cells. 5B (also called FIG. 5B) is a confocal image showing the hCD87-CAR-T cell proliferation induced by hCD87 antigen protein. 5C (also called FIG. 5C) is a MTT detection result chart showing the hCD87-CAR-T cell proliferation induced by hCD87 antigen protein.

FIG. 6: 6A (also called FIG. 6A) is a flow cytometry image showing the specifically killing effect of patient's primary hCD87-CAR-T cells on the patient's own non-function pituitary adenoma tumor cells; CD87-CAR-T+NFPA tumor cells. 6B (also called FIG. 6B) is a confocal microscope image showing the specifically killing effect of patient's primary hCD87-CAR-T cells on the patient's own non-function pituitary adenoma tumor cells; NFPA primary tumor cells.

FIG. 7 is an experimental result chart showing the effector-target ratio of the patient's primary hCD87-CAR-T cells on the pituitary adenoma NFPA, PRL and Cushing tumor cells; optical density value (OD) at 490 nm.

FIG. 8: A (also called FIG. 8A) is a luciferase detection image showing the treatment results of the patient's primary hCD87-CAR-T cell models on a human non-function pituitary adenoma tissue mass NOD-SCID transplanted tumor; B (also called FIG. 8B) is a chart showing the direct measurement results of masses after treating the patient's own non-function pituitary adenoma tissue mass NOD-SCID subcutaneous transplanted tumor with the patient's primary hCD87-CAR-T cell model; tumor volume.

FIG. 9: A (also called FIG. 9A) is a luciferase detection image showing the results of the patient's primary hCD87-CAR-T cell model in vitro co-culturing with the primary human pituitary prolactinoma cells; B (also called FIG. 9B) is a luciferase detection image showing the result of treating patient's own pituitary prolactinoma tissue mass NOD-SCID transplanted tumor with the patient's primary hCD87-CAR-T cell model; C (also called FIG. 9C) is a chart showing the direct measurement results of masses after treating the patient's own pituitary prolactinoma tissue mass NOD-SCID subcutaneous transplanted tumor with the patient's primary hCD87-CAR-T cell model; tumor volume.

FIG. 10: A (also called FIG. 10A) is a luciferase detection image showing the results of treating the patient's own pituitary adrenalotropic hormone adenoma tissue mass NOD-SCID transplanted tumor with the patient's primary hCD87-CAR-T cell model; B (also called FIG. 10B) is a chart showing the direct measurement results of masses after treating the patient's own ACTH tissue mass NOD-SCID subcutaneous transplanted tumor with the patient's primary hCD87-CAR-T cell model; tumor volume.

### DETAILED DESCRIPTION AND PREFERRED EMBODIMENTS OF THE INVENTION

In order to treat the non-function pituitary adenoma, pituitary prolactinoma, and pituitary adrenalotropic hormone adenoma, the present invention provides a chimeric antigen the antigen receptor being incorporated in a plasmid carried by a lentivirus.

The present invention used genetic engineering technology to obtain a fusion sequence encoding a chimeric antigen receptor hCD87-CAR. Such fusion sequence was inserted into a vector, packaged into a lentivirus, and then transfected to the human T cells so as to enable the T cells to express the chimeric antigen receptor hCD87scFv. Such tumor T cells expressing chimeric hCD87scFv can recognize the antigen receptor hCD87 on the tumor surface, specifically kill the hCD87-positive tumor cells, and thereby can be used for the treatment of tumors.

The hCD87 antigen gene is over-expressed on the surface of pituitary adenoma cell membrane, while its level of expression is extremely low in normal tissues according to an analysis on peficancerous tissue data in the gene expression spectrum data sets GSE3526, GSE7307 and TCGA database of the human normal tissues, which is almost the same as the background signal in a gene chip. This illustrates that hCD87 antigen has a good expression specificity in the pituitary adenomas.

Unless specifically defined in the present application, all technical terms or abbreviations used herein have the same meanings and scopes as those known to the ordinary skilled in the art.

As used in the present application:

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are contiguous with each other, e.g., are in the same polypeptide chain (e.g., comprise a chimeric fusion protein). In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule as defined below. In one aspect, the costimulatory molecule is chosen from the costimulatory molecules described herein, e.g., 4-1BB (i.e., CD137), CD27 and/or CD28. In one aspect, the CAR comprises a chimeric fusion protein comprising an extracellular antigen binding domain, a transmembrane domain and an intracellular signaling domain comprising a functional signaling domain derived from a stimulatory molecule. In one aspect, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one aspect, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

As used herein, the term “CD87” refers to the plasminogen activator, urokinase receptor, which is an antigenic determinant detectable on pituitary adenoma cells. The human and murine amino acid and nucleic acid sequences can be found in a public database, such as GenBank, UniProt and Swiss-Prot. For example, the amino acid sequence of human CD87 can be found as UniProt/Swiss-Prot Accession No. Q03405 and the nucleotide sequence encoding of the human CD87 can be found at Accession No. NM_001005376.2.

“CAR structure” means _a peptide of the Chimeric Antigen Receptor

CD8a hinge region means CD8a molecule hinge region. As used herein, the term “CD8a” refers to the CD8a molecule. The human and murine amino acid and nucleic acid sequences can be found in a public database, such as GenBank, UniProt and Swiss-Prot. For example, the amino acid sequence of human CD8a can be found as UniProt/Swiss-Prot Accession No. P01732 and the nucleotide sequence encoding of the human CD8a can be found at Accession No. NM_001145873.1. The CD8 antigen is a cell surface glycoprotein found on most cytotoxic T lymphocytes that mediates efficient cell-cell interactions within the immune system. The CD8 antigen acts as a coreceptor with the T-cell receptor on the T lymphocyte to recognize antigens displayed by an antigen presenting cell in the context of class I MHC molecules.

As used herein, the term “CD28” refers to the CD28 molecule. The human and murine amino acid and nucleic acid sequences can be found in a public database, such as GenBank, UniProt and Swiss-Prot. For example, the amino acid sequence of human CD28 can be found as UniProt/Swiss-Prot Accession No. P10747 and the nucleotide sequence encoding of the human CD28 can be found at Accession No. NM_001243077.1. The protein encoded by this gene is essential for T-cell proliferation and survival, cytokine production, and T-helper type-2 development.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank accno. AAA62478.2, or the equivalent residues from a non-human species, e.g., mouse, rodent, monkey, ape and the like. In one aspect, the “4-1BB costimulatory domain” is the sequence provided as SEQ ID NO:16 or the equivalent residues from a non-human species, e.g., mouse, rodent, monkey, ape and the like.

The term “zeta” or alternatively “zeta chain”, “CD3-zeta” or “TCR-zeta” is defined as the protein provided as GenBan Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In one aspect, the “zeta stimulatory domain” or a “CD3-zeta stimulatory domain” is the sequence provided as SEQ ID NO:17. In one aspect, the “zeta stimulatory domain” or a “CD3-zeta stimulatory domain” is the sequence provided as SEQ ID NO:43.

As used herein, the term “IL12” refers to the interleukin 12 molecule. The human and murine amino acid and nucleic acid sequences can be found in a public database, such as GenBank, UniProt and Swiss-Prot. For example, the amino acid sequence of human IL12 can be found as UniProt/Swiss-Prot Accession No. P29460 and the nucleotide sequence encoding of the human IL12 can be found at Accession No. NM 002187.2. This gene encodes a subunit of interleukin 12, a cytokine that acts on T and natural killer cells, and has a broad array of biological activities. Interleukin 12 is a disulfide-linked heterodimer composed of the 40 kD cytokine receptor like subunit encoded by this gene, and a 35 kD subunit encoded by IL12A.

293T Cells: Human Embryonic Kidney 293T Cells.

hCD87scFv is a fragment of an anti-human CD87 monoclonal antibody.

The gene sequence of the chimeric antigen receptor hCD87-CAR may be named as “hCD87-CAR gene structure” or “hCD87-CAR gene sequence” in various locations of the present application, which is a chimeric peptide. Such chimeric peptide may be made by any genetic engineering or molecular cloning technology known to those ordinary skilled in the art.

The hCD87-CAR gene structure is used to modify T cells, so as to make the modified T cells express the chimeric antigen receptor hCD87scFv.

The present invention also provides a nucleic acid sequence encoding hCD87scFv.

Further, as the chimeric antigen receptor hCD87-CAR gene structure, the hCD87scFv comprises a light chain variable region and a heavy chain variable region of the anti-human CD87 monoclonal antibody anti-human CD87 monoclonal antibody; the amino acid sequence of the heavy chain variable region is SEQ ID NO. 1 shown in the Sequence Listing, and the amino acid sequence of the light chain variable region is SEQ ID NO. 2 shown in the Sequence Listing.

The above-mentioned hCD87scFv can be expressed on the surface of T cells by a CAR structure, where the T cells having hCD87scFv expression may be introduced into tumor cells. The CAR structure is a gene for ensuring the T cells to survive and proliferate in vivo. The CAR structure can activate T cells and make T cells proliferate while specifically enable T cells to be bound to and kill tumor cells. The CAR structure also allows T cells to have memory for removal of reoccurrence of any tumor cells.

According to the present invention, the codons of hCD87scFv are optimized, and the optimized DNA fragment is inserted into a framework plasmid to form a lentiviral expression hCD87scFv vector, i.e., a hCD87scFv expression plasmid. Such expression plasmid may be packaged into 293T cells using a lentivirus packaging plasmid to form a complete lentiviral vector comprising hCD87-CAR. The complete lentiviral vector may be used to infect T cells to enable such infected T cells to express the scFv of the chimeric antigen receptor hCD87.

The nucleic acid sequence encoding the heavy chain variable region may be SEQ ID NO. 3 shown in the Sequence Listing.

The nucleic acid sequence encoding the light chain variable region may be SEQ ID NO. 4 shown in the Sequence Listing.

Said gene structure of the chimeric antigen receptor hCD87-CAR is formed by connecting the sequence of chimeric antigen receptor hCD87scFv and the sequence of a CAR in a linear fashion.

The CAR structure or sequence comprises a CD8a hinge region, CD28, 4-1BB, CD3z and IL12, and the amino acid sequence of the CD8a hinge region may be SEQ ID NO. 5 shown in the Sequence Listing.

The CAR structure comprises a CD8a hinge region, CD28, 4-1BB, CD3z and IL12, and the nucleic acid sequence of the CD8a hinge region may be SEQ ID NO. 6 shown in the Sequence Listing.

The amino terminal of the hCD87scFv comprises a CD8a signal peptide, and the amino acid sequence of the CD8a signal peptide may be SEQ ID NO. 7 shown in the Sequence Listing.

The amino terminal of the hCD87scFv comprises a CD8a signal peptide, and a nucleic acid sequence encoding the CD8a signal peptide may be SEQ ID NO. 8 shown in the Sequence Listing.

In hCD87scFv, there is a connecting peptide between the heavy chain variable region and the light chain variable region. The amino acid sequence of the connecting peptide between the heavy chain variable region and the light chain variable region is exemplified by SEQ ID NO. 9 shown in the Sequence Listing, but may vary to the extent that will not materially change the structure and function based on the relevant knowledge of an ordinary skilled in the art.

Further, the nucleic acid sequence of the connecting peptide between the heavy chain variable region and the light chain variable region may also be exemplified by SEQ ID NO. 10 shown in the Sequence Listing, but may vary to the extent that will not materially change the its structure and function based on the relevant knowledge of an ordinary skilled in the art.

The nucleic acid sequence encoding IL12 and a connecting sequence may be SEQ ID NO. 13 shown in the Sequence Listing.

The hCD87-CAR gene structure is consisted of CD8a signal peptide, the heavy chain variable region of hCD87scFv, a connecting peptide, the light chain variable region of hCD87scFv, a connecting peptide, CD8a hinge region, CD28, 4-1BB, CD3z, a connection peptide, and IL12 (interleukin) constructed in a linear fashion.

The amino acid sequence of the hCD87-CAR may be SEQ ID NO. 11 shown in the Sequence Listing.

The nucleic acid sequence of the hCD87-CAR may be SEQ ID NO. 12 shown in the Sequence Listing.

The present invention also provides a plasmid, comprising the hCD87-CAR gene sequence.

The present invention also provides a lentivirus, comprising the hCD87-CAR gene sequence.

The present invention also provides an application of hCD87-CAR in preparation of hCD87-CAR-T cells and an application of hCD87-CAR in preparation of pharmaceuticals for the tumor therapy.

The hCD87-CAR can modify T cells. After introducing the T cells expressing hCD87scFv into tumor cells, such T cells will be activated and proliferate, specifically bind with tumor cells, and produce interleukin 12 (IL12) to kill the tumor cells.

T cells may be transfected by the plasmid comprising hCD87-CAR gene structure, such that hCD87scFv is expressed on the surface of the transfected T cells.

The gene structure of the chimeric antigen receptors hCD87-CAR provided by the present invention can be used to modify T lymphocytes, and the modified T cells (CAR-T cells) can be used for tumor therapy of surface antigen hCD87-positive.

In contrast to prior art technology, the hCD87-CAR gene structure provided by the present invention can modify T cells, and the modified T cells can be used for the treatment of solid tumors, such as pituitary adenomas including non-function pituitary adenoma, pituitary prolactinoma, and pituitary adrenalotropic hormone adenoma.

T cells modified by using the hCD87-CAR gene structure provided by the invention acquired a strong ability to specifically activate an antigen, therefore can effectively kill the tumor cells.

The solutions provided by the present invention are further illustrated in combination with the drawings and the following examples, none of which serves to limit the scope of the present invention.

### Example 1

### Preparation of a hCD87-CAR Gene Structure

A hCD87-CAR structure is formed by human CD8a signal peptide, a heavy chain variable region, a connecting peptide, a light chain variable region, a connecting peptide, CD8a hinge region, CD28, 4-1BB, CD3z, and IL12 in a linear fashion such as hCD87-scFv-CD8a-CD28-4-1BB-CD3z-IL12.

The nucleic acid sequence of the hCD87-CAR gene structure obtained in Example 1 is SEQ ID NO. 12 shown in the Sequence Listing.

### Example 2

The hCD87-CAR gene structure obtained in Example 1 is used to package a lentiviral vector and a control vector, where Lentiviral vector is hCD87-CAR and the control vector is NC-CAR (NC: absent hCD87scFv sequence), respectively. The lentiviral packaging procedure comprises the following steps:

1. preparation of the packaging materials:

Packaging plasmid pMD2.G, psPAX and plasmid: pCDH

293T cells

DMEM complete medium (Hyclone Corporation, USA): 10% FBS, double-antibody

Serum-free medium Opti-MEM (Invitrogen, USA)

Fetal bovine serum FBS (Gibco company, USA, number: 26140-079)

Transfection Reagent: Lipofectamine 2000 (Invitrogen, USA)

Virus concentrating reagent PEG-it (BioVision company, USA, number: K904-50/200)

Fluorescence Microscopy

2. packaging:
(1) Constructing lentivirus framework plasmid vector

1.1 Amplifying the hCD87scFv domain of the hCD87-CAR gene structure provided in Example 1, and the amplification product has BamHI and EcoR I restriction sites;

1.2 The PCR amplification product and pYr-Lvsh vectors were double digested with BamHI and EcoR I, respectively;

1.3 Recovering the digested fragment, connecting, and transforming into E. coli DH5α;

1.4 Extracting the plasmid, and the extracted plasmid was indentified with enzyme digestion. Using Xho I to digest, and the positive plasmid would be cut a small fragment of approximately 2 kb. The plasmid indentified by enzyme digestion to be correct was further subjected to the sequencing identification;

1.5 Sequencing and verifying the plasmid indentified to be correct, and the correct was a recombinant plasmid pCDH-hCD87-CAR;

In this step, a plasmid comprising the hCD87-CAR gene structure was obtained.

(2) 293T cells inoculation

The 293T cells growing well were digested with 0.25% trypsin, so as to prepare a single-cell suspension, and the concentration was adjusted to be 4×10⁵/ml. Taking 10 ml to inoculate into a 10 cm Petri dish, and culturing at 37° C. in a 5% CO₂ incubator overnight. The next day the cells reached 80% confluency.

(3) Lentivirus packaging

Adding 3 μg pMD2G, 6 μg psPAX and 7.5 μg plasmid obtained in step (1) into 150 μl OPTI-MEM and mixing; adding 25 μl Lipo 2000 into 500 μl OPTI-MEM and mixing, keeping still at room temperature for 5 min, and then adding the plasmid mixture slowly into Lipo 2000. After mixing, keeping the mixture still at room temperature for 15 minutes (min). Adding dropwise the mixture into a Petri dish, and mixing fully. After 6 hours (h), changing with a DMEM fresh medium containing 10% FBS. (Plasmid requirements: concentration of 1 μg/μl, the value of purity OD260/OD280 is in the range of 1.8-2.0).

In this step, lentivirus was producing.

(4) Collecting the supernatant and concentrating the virus

After culturing cells for 48 h, detecting the infection effect under a fluorescence microscope, and collecting the medium (including lentivirus). Centrifuging at 1500 g, 4° C. for 10 min to remove cell debris. Then using a 0.45 μm filtration membrane to filter the virus supernatant. The resulting filtration solution was added 5×PEG-it virus concentrate according to a ratio by volume of filtration solution:concentrate=4:1. After keeping still at 4° C. overnight, centrifuging at 3200 g, 4° C. for 20 min. Discarding the supernatant, resuspending the virus pellet with DMEM medium containing 5% FBS. Distributing to 200 μl/tube, storing the aliquots at −80° C. The remaining 10 μl virus was subjected to the titer determination.

In this step, a concentrate solution of lentivirus comprising hCD87-CAR gene structure was obtained.

A control lentivirus vector was also obtained.

### Example 3

The lentiviral vector and control vector provided in Example 2 are used to infect a human Jurkat T cell line or the primary human peripheral blood T cells, respectively.

The groups were set according to the expression of chimeric antigen receptors as follows:

hCD87-CAR-T group,

NC-CAR-T group; (no hCD87scFv)

NS group (saline group).

Preparing the human peripheral blood primary cells or Jurkat hCD87-CAR-T cells:

1. Isolating T cells

2. Activating T cells

2.1 Materials needed

PBS, sterile water, OKT3 (1 mg/ml) (anti-human CD3 monoclonal antibody), CD28 mAb (1 mg/ml), RetroNectin (recombinant human fibronectin), hIL-2, 24-well plate (not a cell culture plate), RPMI1640 complete medium (containing 10% FBS, 100 U/ml ampicillin, 100 μg/ml streptomycin and 2 mmol/1 GlutaMAX-I)

2.2 Experimental steps

On day 0, using antibody to coat a 24-well plate

(1) On a non-cell culture plate, 24-well plate, marking the needed wells;

(2) Antibody Dilution: Diluting the OKT3 and CD28mAb to 1 μg/ml with sterile water (e.g., if 6 wells needs to be packaged, taking 3 ml of sterile water, then adding 3 μl OKT3 and 3 μl CD28 mAb respectively, and mixing);

(3) Adding 0.5 ml antibody dilution in the marked wells;

(4) Using a sealing membrane to close the surrounding of the plate, placing horizontally at 4° C. overnight.

On day 1, activating PBMC

(1) Discarding the antibody coating solution, washing once with 1 ml RPMI1640 culture;

(2) Discarding the culture, adding 2 ml of freshly prepared PBMC to each well (5×10⁵/ml);

(3) Placing the plate in a 37° C., 5% CO₂ incubator to culture for 24 hours.

On day 2, changing the medium with PBMC and using RetroNectin to coat the 24-well culture plate

(1) Changing PBMC and adding IL-2, aspirating 1 ml of culture each well and adding 1 ml of fresh culture RPMI 1640 complete medium (containing 200 U/ml IL-2), and then placing in a 37° C., 5% CO₂ incubator for further culture;

(2) RetroNectin coating, i.e., diluting RetroNectin with PBS to be a concentration of 20 ng/μl, adding 0.5 ml per well, coating a suitable number of wells according to the experimental requirement;

(3) Using a sealing membrane to close the surrounding of the plate, placing horizontally at 4° C. overnight.

3. Using a lentivirus vector containing hCD87-CAR (called as Lenti-hCD87-CAR) to infect and activate T cells

On day 3, using Lenti-hCD87-CAR to infect and activate the human primary T cells or a human Jurkat T cell line

(1) Collecting the activated T cells, i.e., collecting the activated T cells in a 15 ml centrifuge tube, centrifuging at 400 g for 5 min, and using RPMI 1640 complete medium to resuspend the cells, counting, and dividing to five 1.5 ml centrifuge tubes, 400 μl each tube;

(2) Setting different MOI infection groups. In order to obtain the satisfactory infection efficiency, setting up 5 different MOI values (MOI is 1, 5, 10, 20 and 40 respectively); and according to the MOI, T cell number each group and Lenti-hCD87-CAR virus titer (TU), calculating the virus amount to be added for infecting each group;

(3) Virus infection. According to the calculation result, adding corresponding virus solution to each tube, placing the 1.5 ml centrifuge tube in a 37° C. incubator for 30 min;

(4) Transferring the mixture of cells and virus in the 1.5 ml centrifuge tube to the RetroNectin-coated culture wells, adding 1 ml RPMI 1640 complete medium and IL-2 of a final concentration of 100 U in each well;

(5) Placing the 24-well culture plate in a 37° C. incubator and further culturing.

4. Detecting the infection efficiency of a lentiviral vector containing hCD87-CAR

On day 6, detecting the T cells infection efficiency

Detecting the infection efficiency of T cells after infecting the T cells for 72 hours. Since Lenti-hCD87-CAR has a 2A-eGFP sequence, the infection efficiency can be preliminary determined under a fluorescence microscope.

(1) Gently pipetting the cells in the wells, transferring to a 1.5 ml centrifuge tube, then centrifuging at 400 g for 5 min. Discarding the supernatant, then resuspending the cells with PBS;

(2) Taking about 1×10⁶ cells for the confocal microscopy detection, and determining the infection efficiency by GFP signal.

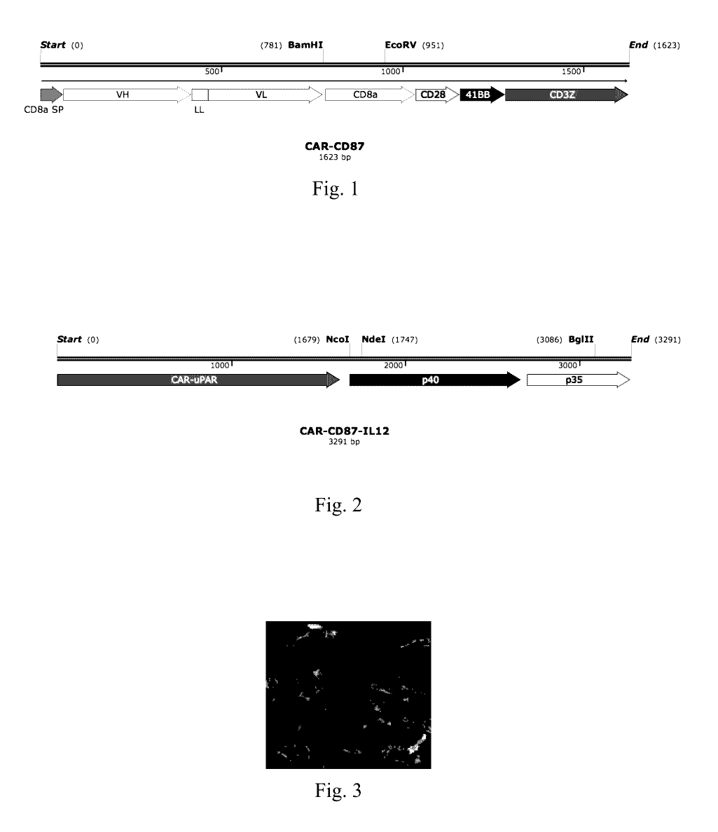

As shown in FIG. 3, under a fluorescence microscope, preliminary determining the infection efficiency, and the infection efficiency of viral to T cells was 62%.

5. Storing a lentiviral vector comprising hCD87-CAR (Lenti-hCD87-CAR)

After harvesting a virus solution, storing it at −80° C. or a lower temperature environment (if storing at 4° C., the virus solution should be used out within one week). For multiple use, the virus solution may be stored after individual packing, so as to avoid repeated freezing and thawing and thereby reduce the viral titer. Usually, the virus can be stored stably for about six months at −80° C. If exceed this period, the virus titer need to be re-detected.

The lentivirus unit is marked as TU/ml, i.e., the number of lentiviral particles having biological activity per milliliter lentiviral solution. For example: if the virus titer is >1×10⁸ TU/ml, i.e. each milliliter of virus solution contains at least 1×10⁸ biologically active virus particles. “TU” is the abbreviation of “Transducing Units”, which represents the number of the viral genome capable of infecting and entering into the target cell population.

6. he sequencing verification of the hCD87-CAR gene structure expression

Taking 1×10⁶ cells, using TRIzoL to extract the total RNA. After synthesizing the first strand of cDNA by reverse transcription, PCR amplifying the gene structure fragment containing the hCD87-CAR. The upstream primer of the hCD87scFv DNA fragment was 5′-CCGCCTTGCTCCTGCCGCTGGCCTT-3′, which was SEQ ID NO. 14 shown in the Sequence Listing; and the downstream primer was 5′-CAGGAAGACCGGCACGAAGGATCCG-3′, which was SEQ ID NO. 15 shown in the Sequence Listing; the amplified product was 787 bp.

After the direct sequencing, comparing the sequence of the amplified fragment, the comparison result proved that the hCD87-CAR-T cell model was correct.

### Example 4

Using the plasmid obtained in the step (1) of Example 2 to directly transfect the primary human T cells, thereby obtaining the human primary hCD87-CAR-T cells.

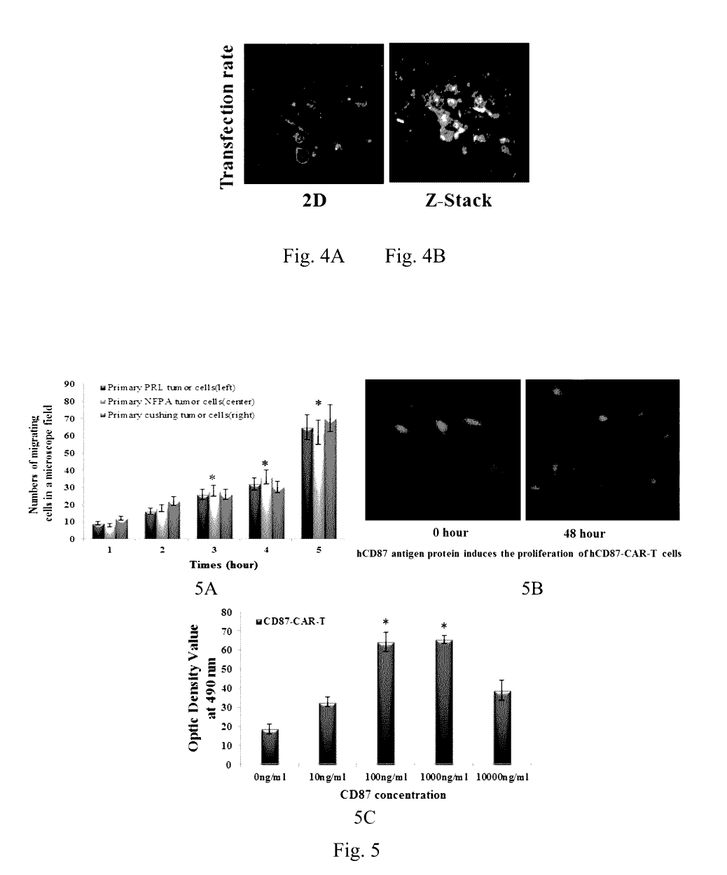

As shown in FIG. 4A and FIG. 4B, the efficiency of the plasmid transfecting T cells was 20%.

### Example 5

Utilizing the human Jurkat hCD87-CAR-T cells prepared in Example 3, Transwell detecting the migration action of primary pituitary adenoma tumor cells on the human Jurkat hCD87-CAR-T cells.

The specific method was as follows:

All the cell culture reagents and a Transwell chamber were incubated at 37° C., after the cells to be tested grew for 24 hours, suspending the cells with 10% serum medium 1640, counting, and adjusting the concentration to be 5×10⁵ cells/ml. In Transwell lower chamber (i.e., the bottom of 24-well plate), adding 1.25 ml primary pituitary adenoma tumor cells of the corresponding proportion; in the upper chamber, adding 200 μl cell suspension. Further culturing in the incubator for 10-24 hours. Using a tweezer to carefully remove the chamber (small room), blotting the immobilization liquid in the upper chamber, and moving to a well previously added about 600 μl formaldehyde, immobilizing at room temperature for 5-10 minutes. Removing the chamber, blotting the immobilization liquid, carefully wiping the cells on the membrane surface of the upper chamber bottom with a damp cotton swab, moving to a well previously added about 600 μl trypan blue dye, staining at room temperature for 3-5 minutes. Gently washing and soaking with PBS twice, removing the chamber, sucking the upper chamber liquid, drying upside down; carefully peeling off the membrane with a small tweezer, moving to a slide, coverslipping. Taking four random fields of a microscope for counting, obtaining a statistical result, taking the average value.

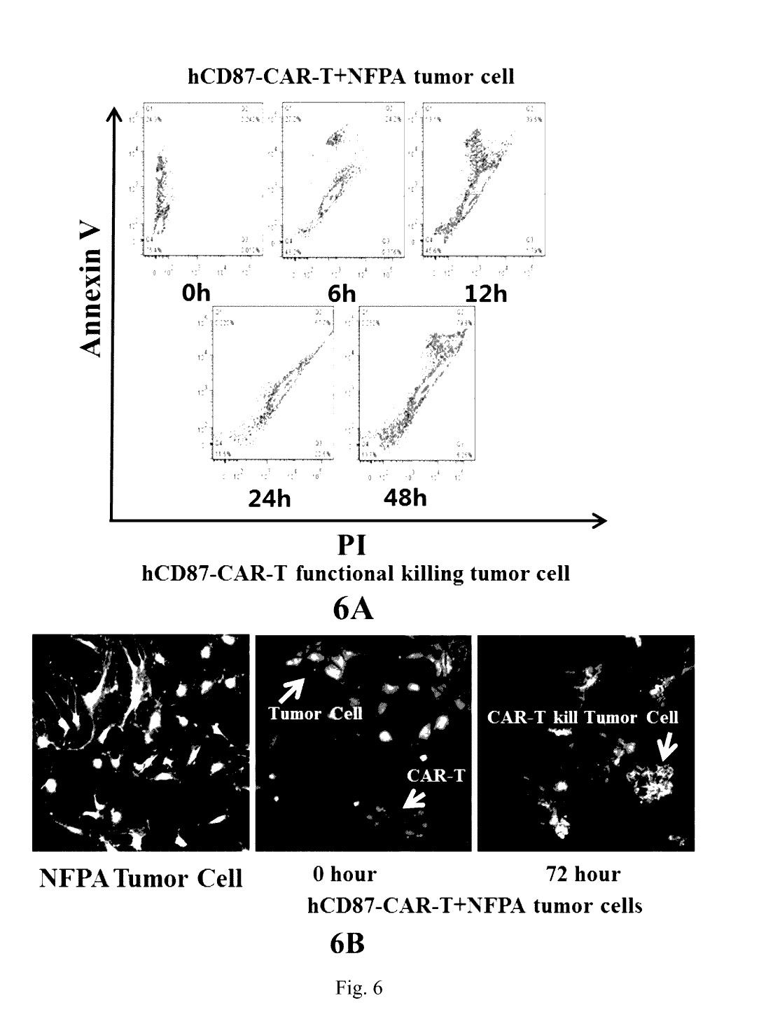

Transwell detecting the inducing migration action of pituitary adenoma cells on the hCD87-CAR-T cells, the detection result was shown in FIG. 5A.

As shown in FIG. 5A, the inducing migration actions of the primary prolactinoma (PRL) tumor cells, primary non-function pituitary adenomas (NFPA) tumor cells, and primary pituitary adrenalotropic hormone adenoma (Cushing) tumor cells among the pituitary adenomas on the hCD87-CAR-T cells were stronger.

Using the human Jurkat hCD87-CAR-T cells prepared in Example 3 to detect the inducing action of the hCD87 antigen protein on the proliferation of Jurkat hCD87-CAR-T cells.

Human Jurkat hCD87-CAR-T cells were inoculated into a 6-well plate, at 1×10⁶ cells/well, keeping still overnight. In each well, adding 1 ml RPMI 1640 complete medium and IL-2 of the final concentration 100 U. In each well, adding antigenic proteins hCD87 of a final concentration 0.1 μM, incubating in a 37° C., 5% CO₂ incubator for 72 hours. In the laser confocal microscope, detecting the fluorescent protein (EGFP) expressing green fluorescence and photographing (FIG. 5B), using the MTT colorimetric assay to detect the induction of the hCD87 antigen protein on the proliferation of Jurkat hCD87-CAR-T cells, and recording the optical density (OD) at 490 nm (FIG. 5C).

As shown in FIG. 5B, the confocal microscopy showed that at 12 hours, the proliferation of the hCD87-CAR-T cells induced by the hCD87 antigen protein was obvious.

As shown in FIG. 5C, the MTT detection of the proliferation of the hCD87-CAR-T cells induced by the hCD87 antigen protein illustrated that, when the concentration of hCD87 antigen protein was 100-1000 ng/ml, the induced hCD87-CAR-T cells proliferation effect detected by MTT was the strongest.

### Example 6

Utilizing the lentivirus prepared in Example 2 to infect the primary T cells isolated from peripheral blood of a patient, thereby obtaining the patient's primary hCD87-CAR-T cells, and detecting the effect of the primary CD87-CAR-T cells specifically killing the patient's own primary non-function pituitary adenoma tumor cells. The patient had signed the informed consent.

In each well of a 6-well plate, adding 1 ml RPMI 1640 complete medium, and primarily culturing the patient's own tumor tissue obtained by a surgery, thereby obtaining the primary human non-function pituitary adenoma cells. Inoculating the cells at 1×10⁶ cells/well into a 6-well plate, incubating in a 37° C., 5% CO₂ incubator for 24 hours. Washing with PBS once, adding 2000 human primary hCD87-CAR-T cells in each well for co-culture. Adding RPMI 1640 complete medium and IL-2 of a final concentration of 100 U, and then incubating in a 37° C., 5% CO₂ incubator for 72 hours. Using laser confocal microscopy and flow cytometry respectively to detect the effect of the patient's primary CD87-CAR-T cells for specifically killing the patient's own primary non-function pituitary adenoma tumor cells.

FIG. 6A and FIG. 6B showed the effect of the patient's primary CD87-CAR-T cells for specifically killing the patient's own primary non-function pituitary adenoma tumor cells.

As shown in FIG. 6A, the flow cytometry showed the effect of the patient's primary CD87-CAR-T cells for specifically killing the patient's own primary non-function pituitary adenoma tumor cells. At 24-48 hours, a large number of tumor cells were apoptotic.

As shown in FIG. 6B, the confocal microscopy showed the effect of the patient's primary CD87-CAR-T cells for specifically killing the patient's own primary non-function pituitary adenoma tumor cells, at 72 hours, a large number of tumor cells died.

### Example 7

Utilizing the lentivirus prepared in Example 2 to infect the primary T cells of different patients, thereby obtaining the primary hCD87-CAR-T cells of different patients. The patients had signed the informed consent.

Detecting the effector to target ratios of the different patients' primary hCD87-CAR-T cell models to their own pituitary adenoma NFPA, PRL or Cushing tumor cells.

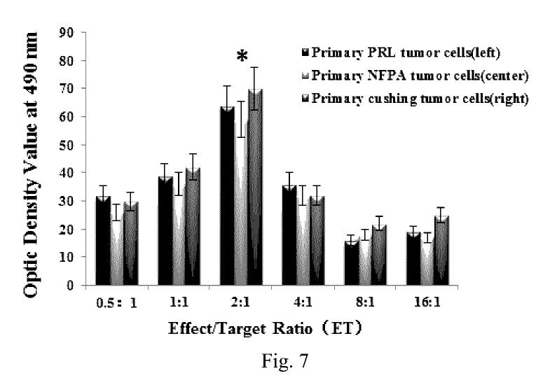

According to the LDH (lactate dehydrogenase) cytotoxicity detection kit, the steps comprised: inoculating 100 μl 1×10⁴ cells/well of target cells (pituitary adenoma cells) into a 96-well plate, adding human primary hCD87-CAR-T cells according to different effector to target ratio ET value (0.5:1, 1:1, 2:1, 4:1, 8:1, 16:1), and supplementing the culture solution up to 200 μl. After co-culturing at 37° C., 5% CO₂ for 72 hours, removing the cell culture plate from the cell incubator, and adding the LDH (lactate dehydrogenase) releasing reagent provided by the kit into the wells inoculated with target cells, the addition amount is 10% of the original culture volume (20 μl). After adding the LDH releasing reagent, repeatedly pipetting for blending several times, and then further incubating in a cell incubator for 1 hour. Centrifuging the cell culture plate with a multiple-well plate centrifuge 400× for 5 min. The supernatant of 120 μl of each well was taken and added into the corresponding well of a new 96-well plate, and then immediately testing the samples. Each well was added 60 μl LDH detection fluid. Mixing, and dark incubating at room temperature of about 25° C. for 30 min, the plate may be wrapped in an aluminum foil and then slowly shaken on a horizontal shaker or a side-to-side shaker, then detecting the optical density at 490 nm wavelength. When the effector to target ratio ET value=2:1, human primary hCD87-CAR-T cells could achieve a good effect of killing the tumor, the effect was about twice that of ET=0.5:1 (see FIG. 7). This in vitro experimental result showed that the constructed human primary hCD87-CAR-T cells had a desired effect of killing tumor.

### Example 8

Utilizing the lentivirus prepared in Example 2 to infect the patient's primary T cells, thereby obtaining the patient's primary hCD87-CAR-T cells. Using the patient's primary hCD87-CAR-T cells to proceed an interference experiment on the patient s own non-function pituitary adenoma severe immunodeficient mouse NOD-SCID tissue mass subcutaneous transplanted tumor model. The severe immunodeficient mice NOD-SCID were bought from Weitonglihua biotechnology company. The patient had signed the informed consent.

The experimental result showed that the constructed patient's primary hCD87-CAR-T cells had a desired effect of killing the non-function pituitary adenoma.

The experimental procedure was as follows:

1. NOD-SCID was anesthetized by intraperitoneal injection with 2% sodium pentobarbital (2 mg/kg), then cutting the fresh tissue comprising the non-function pituitary adenoma cells into 3×3×3 mm³ tissue masses, transplanting the masses into NOD-SCID's left armpit subcutaneous, so as to establish the corresponding human non-function pituitary adenoma surgery tissue mass severe immunodeficient mouse NOD-SCID transplanted tumor model. Making history and image analyses on the 5^{th}, 10^{th}, 15^{th}, 20^{th} and 25^{th} day after the transplantation, respectively. Measuring the subcutaneous lumps in the transplantation sites, determining the tumor mass sizes. When the tumor masses grew to a volume of about 3 cm³, proceeding the experiment of the following step 2.

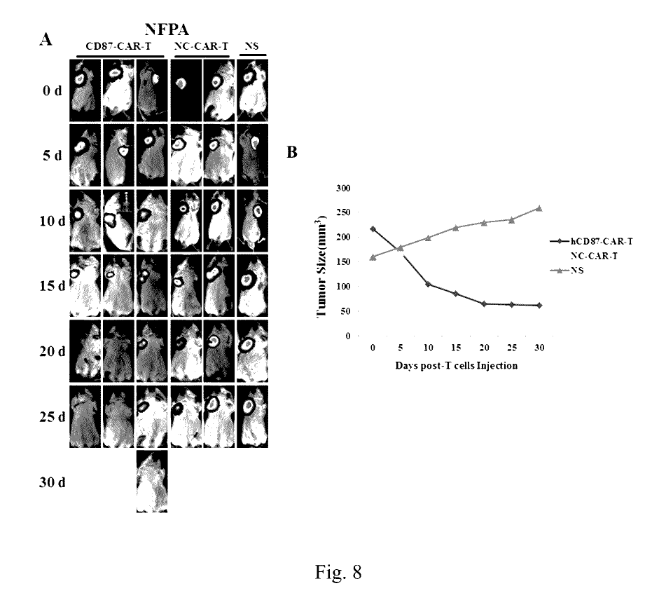

2. NOD-SCID was anesthetized by intraperitoneal injection with 2% sodium pentobarbital (2 mg/kg), then was injected via tail vein with 200 μl 1×10⁶ hCD87-CAR-T cells respectively in the 1^{st}, 2^{nd} and 3^{rd} week after the NOD-SCID transplanted tumor model mass formed. Performing the FX-PRO imaging analysis in vivo on the 5^{th}, 10^{th}, 15^{th}, 20^{th} and 25^{th} day after the first injection. Measuring the subcutaneous lumps at the transplanted sites. The results were shown in FIG. 8A and FIG. 8B.

In FIG. 8B, the ordinate represented the volumes of the tumor masses. The NOD-SCID subcutaneous masses could be measured using vernier caliper, and thereby the volumes of these masses could be obtained.

As shown in FIG. 8A, the patient's primary hCD87-CAR-T cell model in vivo achieved the best effect of specifically killing tumor on the 25^{th} day, the tumor shrank by more than 95%. Whereas the control group had no effects, the reason might lie in that T cells lack hCD87scFv of membrane surface, thus had no strong specifically binding to the tumor antigen hCD87, or failed to effectively activate the tumor-specifically killing action of T cells. As shown in FIGS. 8A and 8B, the above experimental results verified the strong ability of the patient's primary hCD87-CAR-T for in vivo specifically killing non-function pituitary adenoma NOD-SCID transplanted tumor.

### Example 9

Utilizing the patient's primary hCD87-CAR-T cells prepared in Example 4 to proceed the in vitro tumor cell killing experiment and the in vivo NOD-SCID transplanted tumor experiment. The patient had signed the informed consent.

The results showed that patient's primary hCD87-CAR-T could effectively and specifically kill the patient's own pituitary prolactinoma.

Experimental procedure was as follows:

1. NOD-SCID was anesthetized by intraperitoneal injection with 2% sodium pentobarbital (2 mg/kg), then cutting the fresh tissue comprising the pituitary prolactinoma cells into 3×3×3 mm³ tissue masses, transplanting the masses into severe immunodeficient mouse NOD-SCID's left armpit subcutaneous, so as to establish the corresponding human pituitary prolactinoma surgery tissue mass severe immunodeficient mouse NOD-SCID transplanted tumor model. Making history and image analyses on the 5^{th}, 10^{th}, 15^{th}, 20^{th} and 25^{th} day after the transplantation, respectively. Measuring the subcutaneous lumps in the transplantation sites, determining the tumor mass sizes. When the tumor masses grew to a volume of about 3 cm³, proceeding the experiment of the following step 2.

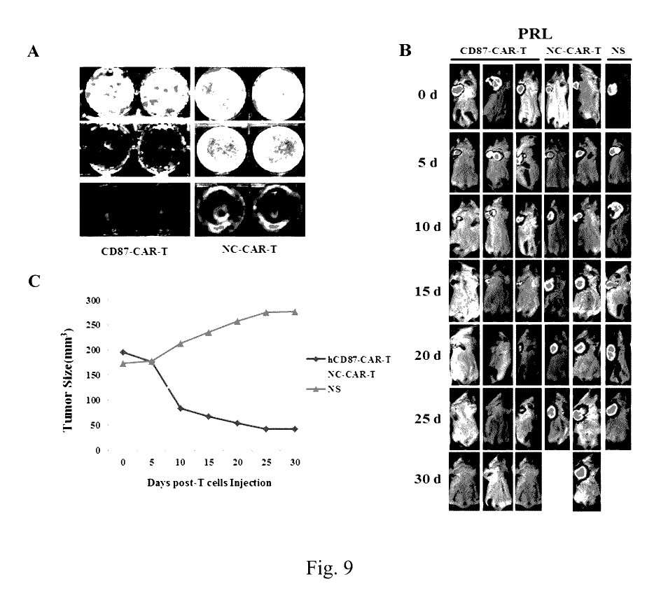

2. NOD-SCID was anesthetized by intraperitoneal injection with 2% sodium pentobarbital (2 mg/kg), then was injected via tail vein with 200 μl 1×10⁶ hCD87-CAR-T cells respectively in the 1st, 2^{nd} and 3^{rd} week after the severe immunodeficient mouse NOD-SCID transplanted tumor model mass formed. Performing the FX-PRO imaging analysis in vivo on the 5^{th}, 10^{th}, 15^{th}, 20^{th} and 25^{th} day after the first injection. Measuring the subcutaneous lumps at the transplanted sites. The results were shown in FIG. 9A and FIG. 9B.

FIG. 9A was a luciferase detection image showing the result of the patient's primary hCD87-CAR-T cell models in vitro co-culturing with the patient's own primary human pituitary prolactinoma cells; the in vitro tumor cell killing experiments was shown in FIG. 9A, the hCD87-CAR-T cells had killing effect on the primary human pituitary prolactinoma cells, which made the primary human pituitary prolactinoma cells die prematurely.

As shown in FIG. 9B, in the human pituitary prolactinoma surgery tissue masses NOD-SCID transplanted tumor model, the patient's primary hCD87-CAR-T cells model in vivo achieved the best effect of specifically killing tumor on the 25^{th} day, the tumor shrank by more than 95%. Whereas the control group had no effects, the reason might lie in that T cells lack hCD87scFv of membrane surface, thus had no strong specific binding to the tumor antigen hCD87, or failed to effectively activate the tumor-specific killing action of T cells. The ordinate in FIG. 9C represented the volumes of the tumor masses. The NOD-SCID subcutaneous masses could be measured using vernier caliper, and thereby the volumes of these masses could be obtained. As shown in FIGS. 9A, 9B and 9C, the patient's primary hCD87-CAR-T cells could effectively and specifically kill tumor. The above experimental results verified the strong ability of the patient's primary hCD87-CAR-T for in vivo specifically killing the patient's own pituitary prolactinomas primary cells.

### Example 10

Utilizing the patient's primary hCD87-CAR-T cells prepared in Example 4 to proceed with the NOD-SCID transplanted tumor experiment. The patient had signed the informed consent.

The results showed that patient's primary hCD87-CAR-T could effectively and specifically kill the pituitary adrenalotropic hormone adenoma.

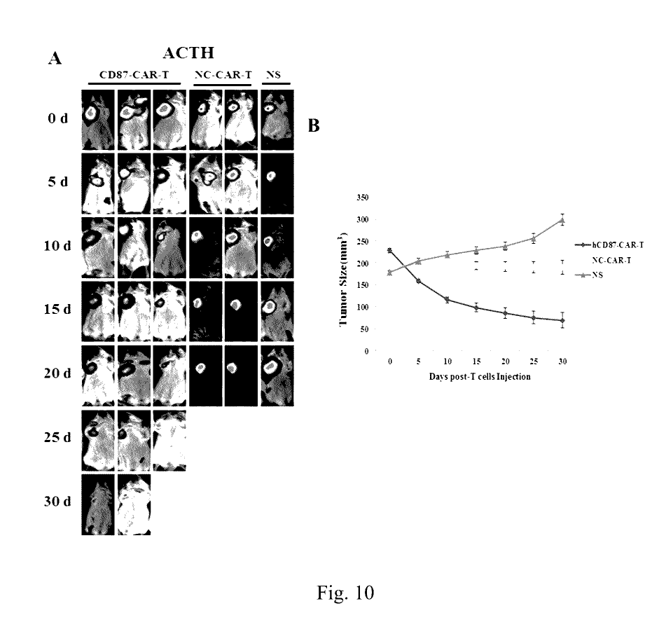

The experimental procedure was the same as that of Example 8, the test results were shown in FIG. 10A and FIG. 10B.

As shown in FIG. 10A, the patient's primary hCD87-CAR-T cells model in vivo achieved the best effect of specifically killing tumor on the 25^{th} day, the tumor shrank by 75%. Whereas the control group had no effects, the reason might lie in that T cells lack hCD87scFv of membrane surface, thus had no strong specific binding to the tumor antigen hCD87, or failed to effectively activate the tumor-specific killing action of T cells. The ordinate in FIG. 10B represented the volumes of the tumor masses. The NOD-SCID subcutaneous masses could be measured using vernier caliper, and thereby the volumes of these masses could be obtained. As shown in FIGS. 10A and 10B, the patient's primary hCD87-CAR-T cells could effectively and specifically kill tumor. The above experimental results verified the strong ability of the patient's primary hCD87-CAR-T for in vivo specifically killing the patient's own pituitary adrenalotropic hormone adenoma cells.

Experimental statistic: Utilizing the lentivirus or plasmid comprising a hCD87-CAR gene structure prepared in Example 2 of the present invention to respectively infect or transfect the T cells of 200 patients, thereby respectively constructing hCD87-CAR-T cell models. Then, using respectively the hCD87-CAR-T cell models to proceed with the NOD-SCID transplanted tumor experiments. The 200 patients were suffering from pituitary adrenalotropic hormone adenoma, or pituitary prolactinoma, or non-function pituitary adenoma. Each of these patients had signed the informed consent. The experimental statistic results were similar to the experimental results in the above Examples 8-10. The experimental statistic results verified the strong ability of the patient's primary hCD87-CAR-T for in vivo specifically killing the patient's own pituitary adrenalotropic hormone adenoma, or pituitary prolactinoma, or non-function pituitary adenoma.

The above examples are o the preferred embodiments of the present invention, which is not intended to limit the scope of the present invention. All equivalent changes and modifications that may be made based on the content of the present invention by those ordinary skilled in the art are encompassed within the scope of the invention patent.

We claim:

1. A peptide of a chimeric antigen receptor namely hCD87-CAR, wherein the chimeric antigen receptor comprises a fragment of anti-human CD87 monoclonal antibody namely anti-human CD87 monoclonal antibody hCD87scFv consisting of a sequence selected from:
(a) SEQ ID NO. 1 or SEQ ID NO. 2;
(b) a sequence having the same function as the sequence of (a); and
(c) a sequence having at least 90% homology to the sequence of (a).

2. A nucleic acid sequence, wherein the nucleic acid encodes the hCD87scFv of claim 1.

3. The peptide according to claim 1, wherein the hCD87scFv comprises a light chain variable region and a heavy chain variable region; the amino acid sequence of the heavy chain variable region being SEQ ID NO. 1, and the amino acid sequence of the light chain variable region being SEQ ID NO. 2.

4. The peptide according to claim 3, wherein the nucleic acid sequence encoding the heavy chain variable region is SEQ ID NO. 3.

5. The peptide according to claim 3, wherein the nucleic acid sequence encoding the light chain variable region is SEQ ID NO. 4.

6. The peptide according to claim 1, wherein the structure is formed by hCD87scFv of claim 1 and a CAR structure in a linear fashion.

7. The peptide according to claim 6, wherein the CAR structure comprises a CD8a hinge region, CD28, 4-1BB, CD3z and IL12, and the amino acid sequence of the CD8a hinge region is SEQ ID NO. 5.

8. The peptide according to claim 6, wherein the CAR structure comprises a CD8a hinge region, CD28, 4-1BB, CD3z and IL12, and the amino acid sequence of the CD8a hinge region is SEQ ID NO. 6.

9. The peptide according to claim 6, wherein the amino terminal of the hCD87scFv comprises a CD8a signal peptide, and the amino acid sequence of the CD8a signal peptide is SEQ ID NO. 7.

10. The peptide according to claim 6, wherein the amino terminal of the hCD87scFv comprises a CD8a signal peptide, and a nucleic acid sequence encoding the CD8a signal peptide is SEQ ID NO. 8.

11. The peptide according to claim 3, characterized in that, in the hCD87scFv, there is a connecting peptide between the heavy chain variable region and the light chain variable region.

12. The peptide according to claim 7, wherein the nucleic acid sequence encoding IL12 and a connecting sequence is SEQ ID NO. 13.

13. A plasmid, comprising the peptide of claim 1.

14. A lentivirus, comprising the peptide of claim 1.

15. A method of preparing CD87-CAR-T cells by using the peptide of claim 1.

16. A method of preparing a pharmaceutical composition for tumor therapy by using the peptide of claim 1.

17. A peptide of a chimeric antigen receptor namely hCD87-CAR according to claim 1, wherein the amino acid sequence of the hCD87-CAR may be SEQ ID NO. 11 shown in the Sequence Listing.

18. A nucleic acid sequence according to claim 2, wherein the nucleic acid sequence of the hCD87-CAR may be SEQ ID NO. 12 shown in the Sequence Listing.
